(19) United States
(12) Patent Application Publication
(10) Pub. No.: US 2010/0041976 A1
(43) Pub. Date: Feb. 18, 2010

(54) CRYOSTAT FOR REDUCED CRYOGEN CONSUMPTION

(76) Inventors: Russell Peter Gore, Abingdon (GB); Edgar Charles Malcolm Rayner, Oxon (GB); Stephen Paul Trowell, Oxon (GB)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/262,614

(22) Filed: Oct. 31, 2008

(30) Foreign Application Priority Data
Nov. 2, 2007 (GB) 0721572.6
Dec. 5, 2007 (GB) 0723788.6

(51) Int. Cl. A61B 5/05; F25B 19/00; H01F 6/04

(52) U.S. Cl. 600/410; 62/51.1; 505/163; 335/216

(57) ABSTRACT

A cryostat has a cryogen vessel retained within an outer vacuum container, a thermally insulating jacket surrounding the outer vacuum container and insulating it from ambient temperature.

### BACKGROUND OF THE INVENTION

1. Field of the Invention

The present invention relates to cryostats including cryogen vessels for retaining cooled equipment such as superconductive magnet coils. In particular, the present invention relates to vacuum chambers provided for reducing heat reaching a cryogen vessel, and to venting arrangements allowing cryogen gas to escape from the cryogen vessel.

2. Description of the Prior Art

FIG. 1 shows a conventional arrangement of a cryostat including a cryogen vessel 12. A cooled superconducting magnet 10 is provided within cryogen vessel 12, itself retained within an outer vacuum chamber (OVC) 14. One or more thermal radiation shields 16 are provided in the vacuum space between the cryogen vessel 12 and the outer vacuum chamber 14. In some known arrangements, a refrigerator 17 is mounted in a refrigerator sock 15 located in a turret 18 provided for the purpose, towards the side of the cryostat. Alternatively, a refrigerator 17 may be located within access turret 19, which retains access neck (vent tube) 20 mounted at the top of the cryostat. The refrigerator 17 provides active refrigeration to cool cryogen gas within the cryogen vessel 12, in some arrangements by recondensing it into a liquid. The refrigerator 17 may also serve to cool the radiation shield 16. As illustrated in FIG. 1, the refrigerator 17 may be a two-stage refrigerator. A first cooling stage is thermally linked to the radiation shield 16, and provides cooling to a first temperature, typically in the region of 80-100K. A second cooling stage provides cooling of the cryogen gas to a much lower temperature, typically in the region of 4-10K.

A negative electrical connection 21a is usually provided to the magnet 10 through the body of the cryostat. A positive electrical connection 21 is usually provided by a conductor passing through the vent tube 20.

For fixed current lead (FCL) designs, a separate vent path (auxiliary vent) (not shown in FIG. 1) is provided as a fail-safe vent in case of blockage of the vent tube 20.

The present invention addresses the consumption of cryogen during transportation of the cryostat, or at any time that the refrigerator 17 is inoperative. When the refrigerator 17 is inoperative, heat from the OVC 14, which is at approximately ambient temperature (250-315K), will flow towards the cryogen vessel 12 by any available mechanism. This may be by conduction through support structures (not illustrated) which retain the cryogen vessel and the radiation shield 16 in position within the OVC; by convection of gases, typically hydrogen, which may be present in the volume between the cryogen vessel 12 and the OVC 14; or by radiation from the inner surface of the OVC. Much attention is typically paid to reducing all of these possible mechanisms for thermal influx. Support structures are made as long and thin as mechanically practicable, and are constructed from materials of low specific heat capacities, to reduce thermal influx by conduction. Care is taken to remove as much gas as possible from the volume between the cryogen vessel and the OVC, although many gases will freeze as a frost on the surface of a cryogen vessel if a very cold cryogen such as helium is in use. One or more thermal radiation shields 16 are provided to intercept thermal radiation from the OVC. Any resultant heating of the thermal radiation shield is removed by the refrigerator 17. Further thermal insulation may be provided, such as the well-known “super-insulation”: multilayered insulation of aluminized polyester sheet, typically aluminized polyethylene terephthalate sheet, played in a layer between the cryogen vessel and the thermal shield 16; or between the thermal shield 16 and the OVC; or both.

In operation, cryogen liquid in cryogen vessel 12 boils, keeping the cooled equipment 10 at a constant temperature, being the boiling point of the cryogen. Refrigerator 17 removes heat from the cryogen gas and the thermal shield 16. Provided that the cooling power of the refrigerator is sufficient to remove any heat generated by the cooled equipment and any heat influx reaching the cryogen vessel, the cooled equipment 10 will remain at its steady temperature, and cryogen will not be consumed.

A difficulty arises during transportation of the cryostat, when the refrigerator is switched off; or at any other time that the refrigerator 17 is inoperative. With the refrigerator inoperative, any heat influx reaching the cryogen vessel, and any heat generated within the cryogen vessel, will cause cryogen liquid to boil. As the refrigerator is inoperative, the boiled-off cryogen cannot be recondensed into liquid, and will vent to atmosphere through vent tube 20 or the auxiliary vent. In the case of superconducting magnets, for example as used in Magnetic Resonance Imaging (MRI) systems, liquid helium is typically used as the cryogen. Liquid helium is expensive, and difficult to obtain in some parts of the world. It is also a finite resource. For these reasons, it is desired to reduce the consumption of helium cryogen during transport or at other times that the refrigerator 17 is not operating.

It is of course possible to transport the cryostat and the equipment 10 at ambient temperature, empty of cryogen. This will avoid the problem of cryogen consumption during transport. However, the equipment 10 and indeed the cryostat itself will need to be cooled on arrival at its destination. Such cooling is a skilled process, and on-site cooling has been found to be very expensive. Furthermore, the quantity of cryogen required to cool the equipment and cryostat from ambient temperature on arrival at an installation site has been found to far exceed current consumption rates over a reasonable transport time. Typical current systems are able to travel for at least 30 days without the refrigerator operating, and without the liquid cryogen boiling dry. This is known as the hold time. It is the aim of the present invention to improve the hold time of a cryostat.

Current known solutions consume approximately 2.5-3.0% of cryogen inventory per day of transit time. On current systems, this may equate to a consumption of 50 liters of liquid helium per day. The present invention aims to reduce this level of consumption, and so increase the hold time, simplifying the logistics of delivering a cooled equipment to a destination and/or reducing the consumption of cryogen.

Known attempts to address this problem have met with difficulties. Some of the known attempts to address this problem will be briefly discussed.

A second thermal radiation shield, concentric with first thermal shield 16 may be provided. This has been found somewhat effective in reducing thermal influx to the cryogen vessel, but has required increased size of OVC, and caused increased manufacturing costs.

A thermally conductive pipe has been run around the thermal shield, carrying escaping cryogen gas. As the gas is at a temperature of about 70-100K, such arrangements serve to cool the thermal shield. This has been somewhat effective at reducing thermal influx to the cryogen vessel. Such an arrangement is described, for example, in U.S. Pat. No. 7,170,377 and UK patent application GB 2 414 536, but has also required increased size of OVC to accommodate the thickness of the conductive pipe. Increased manufacturing costs also resulted from the additional assembly effort, and the material and labor costs of providing the cooling pipes and increasing the size of the OVC.

### SUMMARY OF THE INVENTION

The present invention accordingly aims to provide an improved cryostat which reduces consumption of cryogen during transportation, or at any time when active refrigeration is not present, and does not suffer from the problems of the prior art.

The above object is achieved in accordance with the present invention by a cryostat having a cryogen vessel retained within an outer vacuum container (OVC), an active refrigeration unit that cools the OVC, and a thermally insulating jacket surrounding the OVC and insulating the OVC from ambient temperature.

### DESCRIPTION OF THE DRAWINGS

FIG. 1 shows a conventional arrangement of a cryostat containing a superconducting magnet;

FIGS. 2-4 each show a cryostat carried in a pallet, suitable for application of the present invention; and

FIG. 5 shows an arrangement for cooling an OVC of a cryostat according to an embodiment of the present invention.

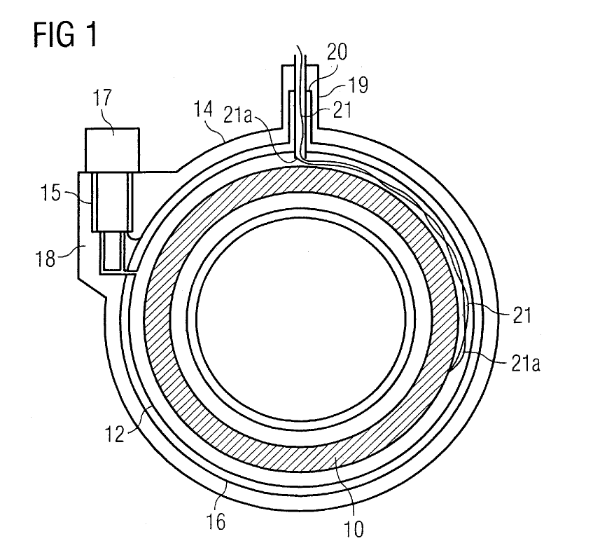

### DESCRIPTION OF THE PREFERRED EMBODIMENTS

The present invention provides reduced consumption of cryogen during transport, or at any time that the active refrigeration is inoperative, by cooling the OVC. Thermal influx to the cryogen vessel 12 takes place by many mechanisms, and most of these mechanisms operate in dependence on the temperature of the outer vacuum chamber.

For example, heat conduction to the cryogen vessel depends upon the thermal conductivity of support structures and other equipment in mechanical connection between the OVC and the cryogen vessel, such as vent tubes 20, electrical connections 21, 21a. However, the heat introduced along each of these conductors depends on the temperature difference between the cryostat and the OVC. By reducing the temperature of the OVC, the amount of heat reaching the cryogen vessel by thermal conduction will reduce.

Heat is also transferred to the cryogen vessel by thermal radiation. Thermal radiation from the OVC is typically intercepted by a thermal shield 16, and removed from the system by the refrigerator when in operation. When the refrigerator is not in operation, the temperature of the thermal shield will rise, and will emit thermal radiation to the cryogen vessel. If the temperature of the OVC is reduced, then the radiation to the shield will reduce; the temperature of the shield will reduce; the radiation from the shield to the cryogen vessel will reduce, and cryogen consumption will reduce. In current cryostats, thermal radiation is the dominant mechanism for heat influx to the cryogen vessel. The radiated power scales as T4, where T is the difference in temperature between the emitting surface and the receiving surface. By reducing the highest temperature—the temperature of the OVC—a significant reduction in radiated power may be achieved.

Some heat may reach the cryogen vessel by convection of gas within the vacuum space between the cryogen vessel and the OVC. Again, if the temperature of the OVC is reduced, the heat influx by this mechanism will reduce.

The inventors have performed simulations demonstrating the effect of a modest reduction of the temperature of the OVC.

Some assumptions were made to make the simulations simple. The geometry simulated is an infinite cylinder, to avoid complications with substantially planar end covers of the OVC 14, the cryogen vessel 12 and the shield 16. Emissivity values consistent with a stainless steel OVC, aluminum shield and aluminum foil-coated cryogen vessel have been used, as these are common materials in use in current cryostats. An OVC mass of 950 kg has been assumed, along with an ambient temperature of 300K.

The effect of a layer of super-insulation placed between the shield and the OVC, has been included. Twenty layers of density 15 layers/cm are assumed, having a room temperature emissivity of 0.04, and a mean temperature determined by the mean of the shield and OVC temperatures.

The conduction of heat through the shield supports has been included. Conducted power varies with OVC and shield temperatures.

The boil-off rate as a function of shield temperature has been determined. It has been assumed that the cooling power to the shield varies linearly with boil-off rate.

Table 1 shows a summary of the output from the simulation. A reduction of 21% in cryogen consumption (boil-off rate), which corresponds to a similar proportionate improvement in hold time can be achieved by a 20 K reduction in OVC temperature.

| | | Con- | | | | |
|---|---|---|---|---|---|---|
| OVC | Shield | duction | Radiation | Total | | Hold time |
| Temp | Temp | load | load | cooling | Boil-off | improvement |
| [K] | [K] | [W] | [W] | [W] | [liters/day] | % |
| 300 | 110 | 1.64 | 9.27 | 10.91 | 40.16 | |
| 290 | 107 | 1.54 | 8.10 | 9.64 | 35.49 | 11.6 |
| 280 | 104 | 1.47 | 7.09 | 8.56 | 31.51 | 21.5 |

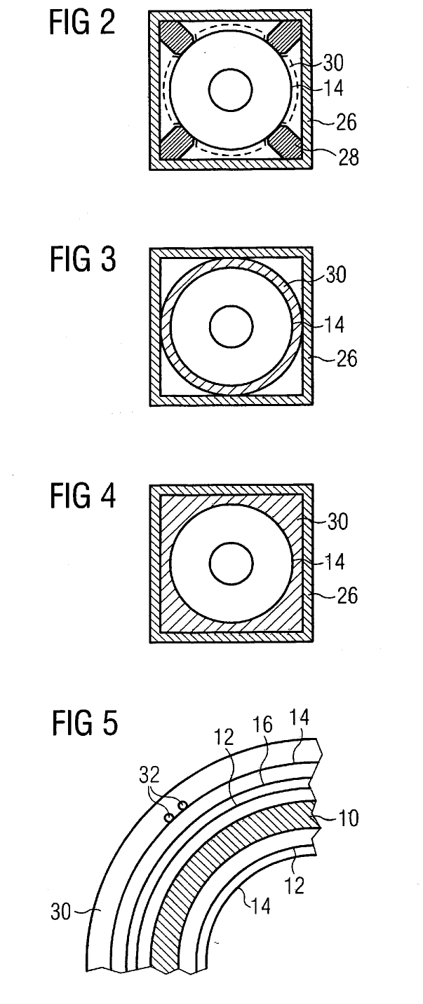

In an example pallet, schematically illustrated in FIG. 2, a thermally insulating jacket 30 is provided around the OVC 14 during transport. The jacket 30 may consist of expanded polystyrene foam or beads, fiberglass, rock wool, wool, bubble wrap, spayed-on polyurethane foam, cloth, super-insulation or any suitable known material for thermal insulation. During transport, thermal radiation from the OVC 14 to the shield 16, and thence to the cryogen vessel 12 will cause the OVC 14 itself to cool, as the radiated heat energy will not be replaced with energy from ambient temperature. Over time, the temperature of the OVC 14 will cool, and the rate of thermal influx to the cryogen vessel 12 will slow, slowing the consumption of cryogen, and extending the hold time.

In an embodiment of the present invention, the jacket 30 is added over the OVC. The OVC is carried in a pallet in the usual manner. FIG. 2 shows an OVC 14 housing cooled equipment (not shown), mounted within a pallet 26, typically an open-sided metal frame, by resilient mounting blocks 28 of rubber or a similarly resilient polymer. The pallet protects the OVC and the cooled equipment from mechanical damage, while the resilient mounting blocks protect the OVC and the cooled equipment from mechanical shocks. Jacket 30 may have holes arranged to allow mounting blocks 28 to pass therethrough, so as not to interfere with the mechanical mounting of the OVC. In such an arrangement, the jacket serves to keep the OVC cool during transport, and may be discarded on arrival at the destination. The pallet may be returned to the supplier for re-use, or may be recycled if this is considered economically viable, or desirable for other reasons. Similarly, the jacket could be returned to the supplier for re-use or recycling. The reduced cryogen consumption during transport obtained by providing the jacket 30 simplifies the logistics of transport, by increasing the length of time during which the OVC and the cooled equipment will remain cold without active cooling. The metal frame making up the pallet may be collapsible, so that once the OVC has arrived at its destination, the pallet may be dismantled, or folded up, to reduce the cost of return transport. The resilient mounting blocks 28 may form part of the pallet, and may be returned to the supplier for re-use with the pallet, or may be returned separately, or may be discarded after arrival at the destination. Similarly, the jacket 30 may form part of the pallet, and may be returned to the supplier for re-use with the pallet, or may be returned separately, or may be discarded after arrival at the destination.

In another pallet, suitable for application of the invention, as illustrated in FIG. 3, the jacket 30 is of a resilient, thermally insulating material such as synthetic foam. By appropriately selecting the material, the thickness and density of the foam, the OVC may rest on the framework of the pallet through the jacket, obviating the need for resilient supports. Alternatively, resilient supports may be integrated into the material of the jacket. In such arrangements, the jacket is preferably returned to the supplier with the pallet, for re-use. The jacket may be removable, such that the pallet may be dismantled to facilitate return shipping. Alternatively, the jacket may not be removable. It may be intended to remain with the OVC after installation. Alternatively, the jacket may be of a material such as a spray-on foam, which is inexpensive to provide, and which is broken off and discarded once the OVC and its cooled equipment have arrived at their destination.

In a particular example, shown in cross-section on FIG. 4, the pallet may be substantially filled with a thermally insulating material 31, with a sufficient cavity left to house the OVC. The OVC will be mechanically restrained within the cavity during transport, and removed on arrival at the destination. In such arrangement, the thermally insulating material provides mechanical protection, protection against mechanical shock and thermal insulation. The metal frame of the pallet may be lighter, since some of the required mechanical strength is provided by the thermally insulating material. It may be found relatively expensive to return such a pallet to the supplier for re-use, but the reduced structural requirement for the metal frame may make single-use disposable pallets of this type economically viable.

The OVC and its cooled equipment may be mounted within a pallet and provided with a thermal jacket. The OVC and its cooled equipment may be transported in the pallet to a further manufacturing site, where further assembly steps are carried out on the OVC and cooled equipment, before it is transported to a final end-user destination, all without leaving the thermally insulating pallet.

In an embodiment of the present invention (not illustrated), an arrangement is made for actively cooling the OVC within the thermally insulating jacket. For example, an electrically powered refrigerator may be provided and employed to cool the OVC within the thermally insulating jacket. Such arrangement may be built into any of the pallets described above, provided that a suitable source of energy, such as an electrical source, is available during shipping, or is built into the pallet.

In a further, preferred, embodiment, illustrated in FIG. 5, cryogen gas escaping from the cryogen vessel is directed through pipes 32 which are located between the OVC 14 and the thermal jacket 30. In response to thermal influx to the cryogen vessel 12, liquid cryogen boils into a cryogen gas, which escapes from the cryogen vessel through vent tube 20 or an auxiliary vent. In the case of helium cryogen, the escaping cryogen gas typically has a temperature of about 70-100K. A thermally conductive pipe 32, for example of copper, is provided between the OVC 14 and the thermally insulating jacket 30, in thermal contact with the OVC 14. By directing at least some of the escaping cryogen gas through the thermally conductive pipe 32, the OVC 14 is cooled. FIG. 5 illustrates one example of this embodiment, where a thermally conductive pipe 32 is in thermal contact with the OVC 14, and the OVC 14 and the pipe 32 are insulated. from ambient temperature by thermally insulating jacket 30. Calculations or trial and error may be performed to determine an optimal length and bore of the pipe. It is preferred that the OVC 14 should be of substantially constant temperature, and so it is envisaged that the thermally conductive pipe 32 should be long enough to encircle the OVC 14 at least once. The thermally conductive pipe may be arranged in a serpentine fashion over an inner or outer surface of the OVC 14; it may be arranged around the outer or inner cylindrical surfaces of the OVC 14, or in. any configuration which provides the desired length of pipe 32 in thermal contact with the OVC 14.

Arrangements must be made to ensure that at least some of the cryogen gas escaping from the cryogen vessel 12 is made to flow through the cooling pipe 32. As will be apparent to those skilled in the art, this may be arranged by a temporary fitting on the vent tube or auxiliary vent.

Such arrangement may be built into any of the pallets described above, or may be permanently affixed to the OVC.

Assuming perfect thermal contact between the helium gas and OVC, no ambient heat load, and helium cryogen gas incident on the OVC at shield temperature (70-100K), the simulation referred to above demonstrates that a 20 K reduction in OVC temperature can be achieved in 2.4 days by use of the boil-off gas enthalpy only.

Typically, the OVC cooling pipe vents the boiled off cryogen gas to atmosphere.

In some embodiments, the cooling pipe 32 may be a permanent fixture, in which case heat transfer between the pipe 32 and the OVC 14 may be improved by bonding the pipe 32 to the OVC 14 by soldering or using a thermally conductive adhesive. It may be found advantageous in such embodiments to provide a permanent thermally insulating jacket, for example of expanded polyurethane foam.

By making the cooling pipe and thermally insulating jacket 30 a permanent feature, the advantages of the present invention may be enjoyed even while the cryostat is in operation, for example containing a superconducting magnet of a magnetic resonance imaging (MRI) system. By thermally insulating the OVC 14 from atmosphere, the temperature of the OVC 14 will be less than ambient, due to the effect of thermal radiation from the OVC 14 to the thermal shield 16, cooled by refrigerator 17. Reduced thermal influx due to reduced OVC 14 temperature may mean that a desired temperature within the cryostat may be achieved with a less powerful refrigerator 17. If cryogen gas escapes during operation and is directed through a cooling pipe 32. of the present invention, the effect will be even more pronounced, and the required power from refrigerator 17 may be reduced still further.

Alternatively, or in addition, arrangements may be made for actively cooling the OVC within the jacket. For example, an electrically powered refrigerator may be provided and employed to cool the OVC within the thermally insulating jacket. Such active refrigeration may be provided by a cooling loop similar to that employed in a domestic refrigerator or freezer.

Some equipment containing a cryostat, such as a magnet in an MRI system, is conventionally provided with “looks” covers, to improve the aesthetic appearance of the cryostat, and to provide acoustic damping. These typically comprise glass-fiber reinforced plastic moldings which are clipped into place over the surface of the cryostat's OVC 14. According to an embodiment of this invention, such looks covers may be provided with thermally insulating material, such as expanded polystyrene or polyurethane foam, or wool, or fiberglass wool, or rock wool, between the surface of the OVC and the “looks” covers themselves. Such thermal insulation may then be a permanent feature of the cryostat in use, and may also provide acoustic damping. In order to provide space for cooling pipes 32, molded channels may be provided in solid insulation such as expanded polystyrene or polyurethane foam. For flexible thermal insulation, such as fiberglass wool, or wool, or rock wool, the insulation may simply deform around the pipes. Other embodiments may include loose material such as expanded polystyrene beads. It is preferred that such material be contained within flexible pouches such as polythene bags to avoid spills. Such thermal insulation would also deform around the OVC cooling pipes.

On installation of the cryostat, the cooling pipes 32 may be left in place, possibly being used during operation of the cryostat by providing an escape path for cryogen gas, or the cooling pipes may be removed. The molded channels which would remain in a molded thermal insulation may be employed to house other components, such as electrical cables.

It may be preferred to remove the cooling pipes 32 on delivery. In such arrangements, a serpentine copper pipe arrangement may be found most advantageous, in that it is flexible enough to be wrapped around the OVC 14. In particular, a serpentine cooling pipe 32 may be wrapped around the outer cylindrical surface of the OVC 14, strapped into place using suitable straps, such as conventional luggage straps, and a flexible thermally insulating jacket 30 may be wrapped and fastened over the cooling pipe 32. The thermally insulating jacket 30 may be of fiberglass, wool, or rock wool enclosed in a suitable outer cover. Alternatively, a serpentine OVC cooling pipe may be retained within a flexible wrapper, such as a thin fiberglass blanket, which may be wrapped around the OVC 14 and tightened to provide sufficient thermal contact between the cooling pipe 32 and the OVC 14. A flexible thermally insulating jacket 30 may then be wrapped and fastened over the blanket.

By making the OVC cooling pipes and thermally insulating jacket temporary fixtures only, the cost of each system may be reduced since the OVC cooling pipes and the thermally insulating jacket may be removed from the cryostat on installation and re-used many times over on other cryostats.

The thermally insulated jacket may be constructed so as to provide mechanical damping to protect the OVC and the cryostat as a whole from mechanical shocks encountered during transport.

The thermally insulated jacket may be constructed so as to protect the OVC and the cryostat as a whole from harmful contaminants which may be encountered during transport, such as seawater.

The thermally insulating jacket may be integrated with a pallet for transporting the system.

In all embodiments of the present invention, extended hold times are enabled by the reduction in thermal influx to the cryogen vessel brought about by a reduction in the temperature of the OVC. In embodiments where the cooling pipe 32 and thermally insulating jacket 30 are removed, there is little manufacturing cost penalty in using the present invention, since the cooling pipe 32 and thermally insulating jacket 30 may be reused several times. In embodiments where a permanent cooling pipe 32 and thermally insulating jacket 30 are provided, the benefits of the present invention may be enjoyed even during operation of the cryostat, by continuing to ensure reduced OVC 14 temperatures. The requirement for later fitting “looks” covers may be avoided, or simplified, by the provision of a permanent thermally insulating jacket.

While the present invention has been described with specific reference to a limited number of particular embodiments, many modifications and variations will be apparent to those skilled in the art, and fall within the scope of the present invention. For example, outer vacuum chambers according to the present invention may be provided in cryostats holding cooled equipment other than magnets for MRI systems, being useful in any cryogenic storage Dewar. Similarly, insulated outer vacuum chambers according to the present invention are useful for cryostats containing any liquid cryogen, and the present invention is not limited to helium-cooled cryostats. While the cooling pipes 32 of the present invention have been discussed as, contacting an external surface of the OVC, the present invention also encompasses. arrangements in which the cooling pipes are provided on an interior surface of the OVC, within the vacuum region between the OVC and the cryogen vessel.

Although modifications and changes may be suggested by those skilled in the art, it is the intention of the inventors to embody within the patent warranted hereon all changes and modifications as reasonably and properly come within the scope of their contribution to the art.

We claim as our invention:

1. A cryostat comprising a cryogen vessel retained within an outer vacuum container (OVC), an active refrigeration unit that cools the OVC, and a thermally insulating jacket surrounding the OVC and insulating the OVC from ambient temperature.

2. A cryostat according to claim 1, wherein the jacket is mechanically bonded to the OVC.

3. A cryostat according to claim 1, wherein the thermally insulating jacket integrates the function of a looks cover.

4. A cryostat according to claim 3, wherein the jacket comprises a solid material having molded channels for accommodating auxiliary equipment.

5. A cryostat according to claim 1, wherein the thermally insulating jacket comprises a flexible material.

6. A cryostat according to claim 1, wherein the thermally insulating jacket comprises a loose material.

7. A cryostat according to claim 6, wherein the loose material is contained within flexible pouches.

8. A cryostat according to claim 1, wherein the thermally insulating jacket provides damping against mechanical shocks experienced by the system during transit.

9. A cryostat according to claim 1, wherein the thermally insulating jacket protects the OVC from contaminants such as sea water during transit.

10. A cryostat according to claim 1, wherein the thermally insulating jacket is integrated with a pallet for transporting the cryostat.

11. A cryostat comprising a cryogen vessel retained within an outer vacuum container (OVC), the cryogen vessel having a vent path allowing cryogen gas to escape from the cryogen vessel, and a thermally insulating jacket surrounding the OVC and insulating the OVC from ambient temperature, and a cooling pipe in thermal contact with the OVC, that directs cryogen gas escaping through the vent path through the cooling pipe, to cool the OVC.

12. A cryostat according to claim 11, wherein the cooling pipe is interposed between the thermally insulating jacket and the OVC.

13. A cryostat according to claim 12, wherein the cooling pipe is detachable from the OVC.

14. A cryostat according to claim 11, wherein the cooling pipe encircles the OVC at least once.

15. A cryostat according to claim 11, wherein the cooling pipe follows a serpentine path over a surface of the OVC.

16. A cryostat according to claim 11, wherein the cooling pipe is flexible, and is contained within a flexible wrapper, which is wrapped around the OVC and overlain with the jacket.

17. A cryostat according to claim 11, wherein the cooling pipe is applied to a cylindrical surface of the OVC.

18. A cryostat according to claim 10, wherein the cooling pipe is mechanically bonded to the OVC.

19. A cryostat according to claim 11, wherein the jacket (30) is mechanically bonded to the OVC.

20. A cryostat according to claim 11, wherein the thermally insulating jacket integrates the function of a looks cover.

21. A cryostat according to claim 20, wherein the jacket comprises a solid material having molded channels for accommodating auxiliary equipment.

22. A cryostat according to claim 11, wherein the thermally insulating jacket comprises a flexible material.

23. A cryostat according to claim 11, wherein the thermally insulating jacket comprises a loose material.

24. A cryostat according to claim 23, wherein the loose material is contained within flexible pouches.

25. A cryostat according to claim 11, wherein the thermally insulating jacket provides damping against mechanical shocks experienced by the system during transit.

26. A cryostat according to claim 11, wherein the thermally insulating jacket protects the OVC from contaminants such as sea water during transit.

27. A cryostat according to claim 11, wherein the thermally insulating jacket is integrated with a pallet for transporting the cryostat.

28. A superconducting magnet assembly comprising a cryogen vessel retained within an outer vacuum container (OVC), an active refrigeration unit that cools the OVC, a thermally insulating jacket surrounding the OVC and insulating the OVC from ambient room temperature, and a superconducting magnet contained within said cryogen vessel.

29. A magnetic resonance imaging system comprising:
a magnetic resonance data acquisition device configured to interact with a patient to acquire magnetic resonance data therefrom, said magnetic resonance data acquisition device having a superconducting magnet configured to receive the patient therein and a cryostat comprising a cryogen vessel, in which said superconducting magnet is contained, said cryogen vessel being retained within an outer vacuum container (OVC) and said cryostat further comprising an active refrigeration unit that cools the OVC, and a thermally insulating jacket surrounding the OVC and insulating the OVC from ambient temperature.
